(19) United States
(12) Patent Application Publication
(10) Pub. No.: US 2022/0355304 A1
(43) Pub. Date: Nov. 10, 2022

(54) MEDICAL UTILITY DEVICE AND METHOD OF USE

(71) Applicant: Gregg Gordon, Somers, NY (US)

(72) Inventor: Gregg Gordon, Somers, NY (US)

(21) Appl. No.: 17/738,598

(22) Filed: May 6, 2022

Related U.S. Application Data
(60) Provisional application No. 63/186,603, filed on May 10, 2021.

(51) Int. Cl. B01L 9/06

(52) U.S. Cl. CPC B01L 9/06; B01L 2300/08

(57) ABSTRACT

A medical utility device, including a unitary body having a base surface, an upper surface, and an intermediate surface, and a plurality of receptacles configured to removably engage a portion of a collection tube in connection with a venipuncture procedure.

### CROSS-REFERENCE TO RELATED APPLICATIONS

This application is related to Ser. No. 63/186,603, filed May 10, 2021, titled “MEDICAL UTILITY DEVICE AND METHOD OF USE,” which is hereby expressly incorporated herein in its entirety including the specification, claims, drawings and abstract.

### STATEMENT REGARDING FEDERALLY SPONSORED RESEARCH

Not applicable.

### BACKGROUND

The subject matter herein relates generally to a medical utility device, and, more particularly, to a medical utility apparatus and method of use in connection with a venipuncture procedure.

Medical and healthcare personnel often collect samples or specimens of bodily fluids, such as blood, from patients for diagnostic purposes. Generally, medical personnel collect blood into collection tubes by puncturing a vein with a needle. For example, a phlebotomist or other personnel generally may obtain blood from a vein, also referred to as venipuncture procedure, from a patient using a needle or other extraction or drawing device, such as a butterfly needle. The extraction device is connected to tubing that can be inserted or attached to a storage device, such as an evacuated collection tube, for transfer and storage of the specimen. For example, a butterfly needle may insert or pierce a rubber stopper of a glass evacuated collection tube to transfer a blood specimen from the patient through the tubing and into the tube. After the desired specimen is obtained, the needle is removed from the tube, and the tube is used for storage and transport of the specimen.

Due to the cost of labor and supplies, it is important that collection, handling, storage, and transport of the specimen are performed efficiently, safely, and accurately to prevent loss, destruction, or contamination of the specimen. In particular, these characteristics are important when multiple specimens are taken from a patient in a single procedure. Conventional devices and methods are clumsy, inefficient, and have a high risk of breaking a collection tube, such as by rolling off a table. In addition, handling individual collection tubes increases the chance of accidental needle sticks.

Therefore, what is needed is a medical utility device that provides safe, efficient handling, storage and transport of collection tubes in connection with a venipuncture procedure.

### BRIEF DESCRIPTION OF THE INVENTION

In one embodiment, a medical utility device is provided that includes a unitary body having a base surface, an upper surface, and an intermediate surface, and a plurality of receptacles configured to removably engage a portion of a collection tube in connection with a venipuncture procedure.

In another embodiment, a medical utility device is provided that includes a unitary body having a base surface, an upper surface and an intermediate surface extending between the base surface and the upper surface, a plurality of receptacles configured to removably engage a portion of a collection tube in connection with a venipuncture procedure, wherein the plurality of receptacles are configured to engage the collection tube in an upright position, and wherein the unitary body is configured for engagement by a user in connection with a venipuncture procedure.

In yet another embodiment, a method of storage for a plurality of collection tubes includes providing a medical utility device having a unitary body having a base surface, an upper surface and an intermediate surface extending between the base surface and the upper surface, and a plurality of receptacles configured to removably engage a portion of a corresponding collection tube in connection with a venipuncture procedure, and collecting a blood specimen into at least one of the plurality of collection tubes.

### BRIEF DESCRIPTION OF THE DRAWINGS

The present inventive subject matter will be better understood from reading the following description of non-limiting embodiments, with reference to the attached drawings, wherein below:

FIG. 1 is a perspective view of a medical utility device engaged with a plurality of collection tubes;

FIG. 2 is a side view of the medical utility device with a collection tube removed;

FIG. 3 is a perspective view of the medical utility device;

FIG. 4 is a top view of the medical utility device;

FIG. 5 is a side view of the medical utility device;

FIG. 6 is a bottom view of the medical utility device;

FIG. 7 is a perspective view of a medical utility device engaged with the plurality of collection tubes and an extraction device;

FIG. 8 is a perspective view of the medical device engaged with a plurality of collection tubes and the extraction device engaged with one of the collection tubes;

FIG. 9 is a perspective view of the medical device engaged with a plurality of collection tubes and the extraction device engaged with another one of the collection tubes;

FIG. 10 is a perspective view of a user holding medical device engaged with the plurality of collection tubes; and

FIG. 11 is a perspective view of a medical device engaged with the plurality of collection tubes resting on a surface.

Corresponding reference numerals indicate corresponding parts throughout the several figures of the drawings.

### DETAILED DESCRIPTION OF THE INVENTION

The following detailed description illustrates the inventive subject matter by way of example and not by way of limitation. The description enables one of ordinary skill in the art to make and use the inventive subject matter, describes several embodiments of the inventive subject matter, as well as adaptations, variations, alternatives, and uses of the inventive subject matter. Additionally, it is to be understood that the inventive subject matter is not limited in its application to the details of construction and the arrangements of components set forth in the following description or illustrated in the drawings. The inventive subject matter is capable of other embodiments and of being practiced or being carried out in various ways. Also, it is to be understood that the phraseology and terminology used herein are for the purpose of description and should not be regarded as limiting on all embodiments of the inventive subject matter.

The terminology used herein is for the purpose of describing particular exemplary embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The steps, processes, and operations described herein are not to be construed as necessarily requiring their respective performance in the particular order discussed or illustrated, unless specifically identified as a preferred order of performance. It is also to be understood that additional or alternative steps may be employed.

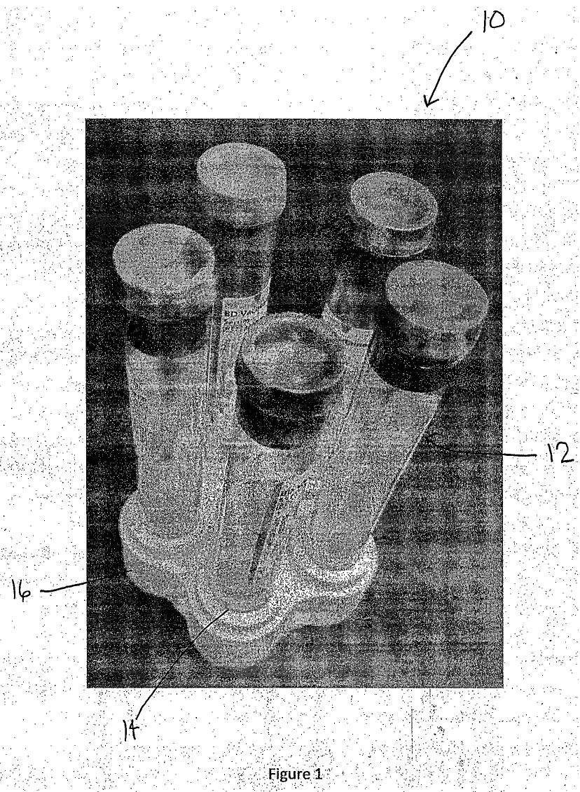

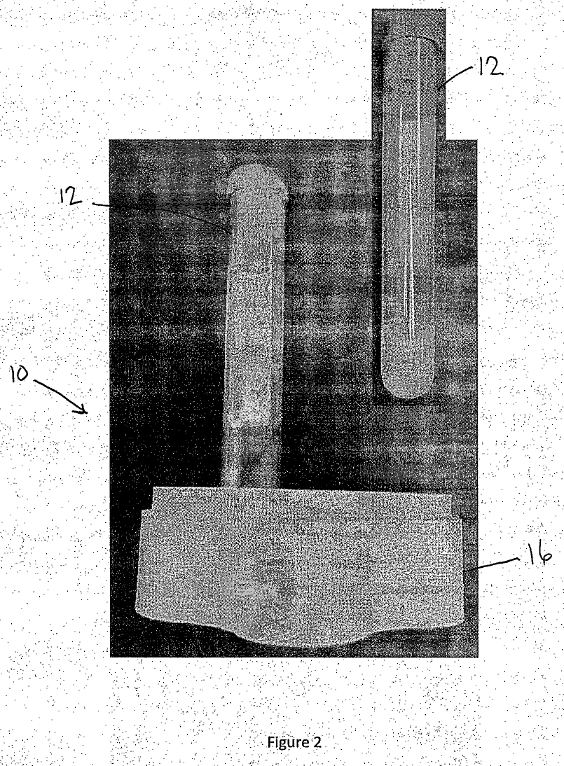

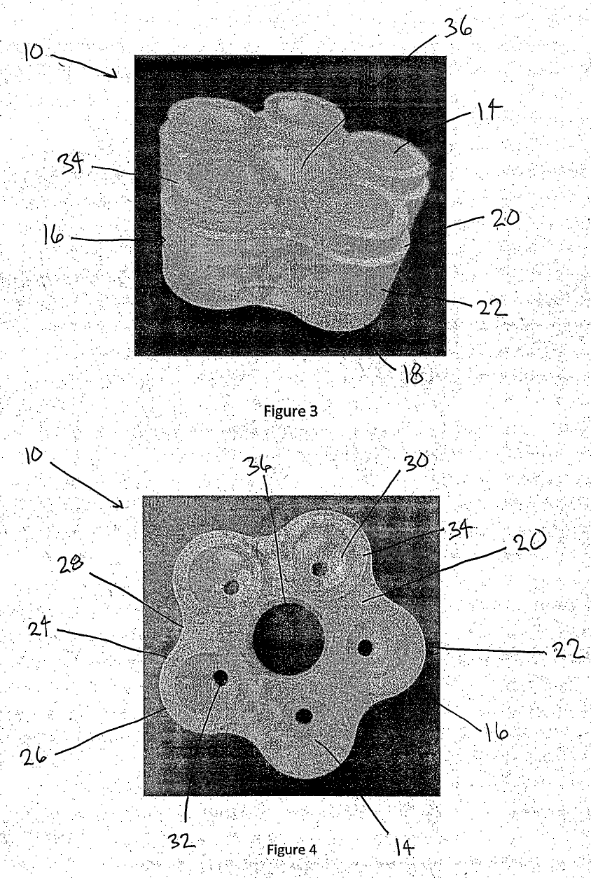

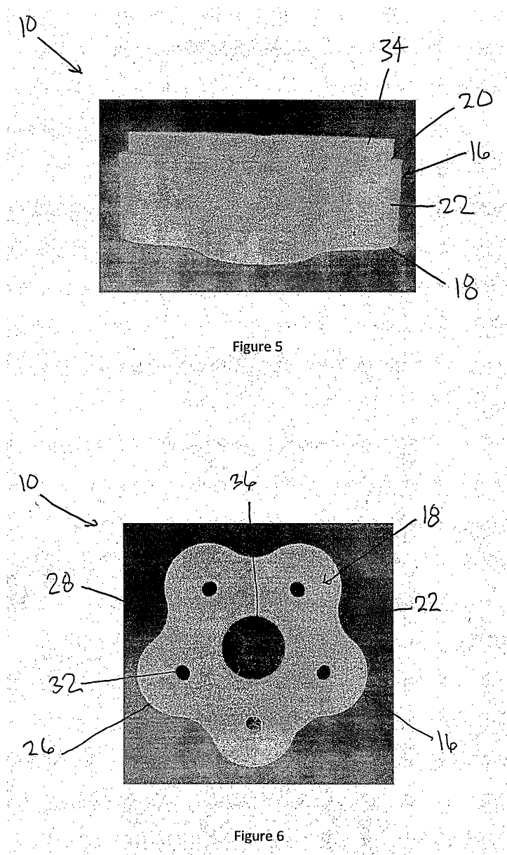

As shown in FIGS. 1-6, an embodiment constructed in accordance with the present disclosure, generally referred to as a medical utility device 10. FIG. 1 is a perspective view of a medical utility device 10 engaged with a plurality of collection tubes 12. FIG. 2 is a side view of the medical utility device 10 with a collection tube 12 removed. In an exemplary embodiment, the medical utility device 10 includes a plurality of receptacles 14 that are configured for removable engagement with a corresponding plurality of collection tubes 12 in connection with a venipuncture procedure (FIG. 3). In the illustrated embodiment, the collection tube 12 is a Vacutainer® blood collection tube manufactured by BD medical. However, any suitable collection tube can be used.

FIG. 3 is a perspective view of the medical utility device 10. FIG. 4 is a top view of the medical utility device 10. FIG. 5 is a side view of the medical utility device 10. The medical utility device 10 includes a unitary base 16 having a base surface 18, an upper surface 20, and an intermediate surface 22 extending therebetween. The unitary base 16 includes a hub 36 extending through the unitary base 16 along a central axis. The base surface 18 is a generally planar surface that is configured for stable resting on any generally flat or planar surface. The upper surface 20 is generally planar and parallel with the base surface 18.

The intermediate surface 22 extends upwardly from and generally perpendicular to the base surface 18. The intermediate surface 22 defines a perimeter 24 with a pattern that is generally pentagon-shaped with rounded corners 26. The perimeter 24 includes indented portions 28 disposed in between each of the rounded corners 26. In an exemplary embodiment, the indented portions 28 are arcuately shaped and have a radius that is generally equal to the radius of the rounded corners 26. The shape of the intermediate surface 22 provides for ergonomic handling holding and handling by a user. For example, the user's fingers can engage with the indented portions 28 for secure holding and convenient rotation of the medical utility device within the user's hand (FIGS. 7-10).

The unitary base 16 includes the plurality of receptacles 14 that are each configured to removably receive and engage a corresponding collection tube 12. In an exemplary embodiment, the receptacles 14 are generally circular holes that extend downwardly from the upper surface 20 and terminate at a lower end 30 proximate to the base surface 18. As shown, the lower end 30 is generally hemispherical to generally correspond to the shape of a lower end of the collection tube 12. A through-hole 32 extends from the lower end 30 of the receptacle 14 through to the base surface 18. The through-hole 32 provides for airflow so that the collection tubes 12 can be inserted and removed from the receptacle without creating any change in pressure that would interfere with engagement between the receptacles 14 and the collection tubes 12. The receptacles 14 are preferably sized to engage with a lower portion of the collection tubes 12 using a friction fit. However, those skilled in the art will recognize that any method of engagement or mechanical clamping mechanism can be used. A raised rim 34 is located proximate to an upper end of at least one of the receptacles 14. In alternative embodiments, other methods of removably securing the collection tube 12 within the receptacle may be used. In addition, the unitary base can include any number of receptacles 14.

The receptacles 14 are positioned proximate to each rounded corner 26. For example, the center of each receptacle 14 may align with the center of the radius for the corresponding rounded corner 26. Those skilled in the art will recognize that the receptacles can be located at any location in the unitary base. In addition, the unitary base 16 may include any number of receptacles, including one. Although the illustrated embodiment discloses a generally pentagon-shaped perimeter 24, those skilled in the art will recognize that in alternate embodiments the perimeter 24, rounded corners 26, and/or indented portions 28 can be any size, shape, or geometry. For example, the rounded corners 26 may be generally rectilinear shaped, generally arcuately shaped generally symmetrical, generally non-symmetrical, or any combination thereof. The indented portions 28 may be generally rectilinear shaped, generally arcuately shaped generally symmetrical, generally non-symmetrical, or any combination thereof. In addition, the perimeter 24 can include rounded corners 26 of a consistent size, shape and geometry or a plurality of sizes, shapes, and geometries. Also, the perimeter 24 can include indented portions 28 of a consistent size, shape and geometry or a plurality of sizes, shapes, and geometries.

In one or more embodiments, the medical utility device 10 may be manufactured from any suitable material, including but not limited to, polymers, metals, metal alloys, or any combination of suitable materials. In one or more embodiments, the utility device 10 may be manufactured from a thermoplastic polymer material, polyether ether ketone, polysulfone, poly(methyl methacrylate), acrylonitrile butadiene styrene, polylactide, polycarbonate, polybenzimidazole, polyetheretherketone, polyoxymethylene, polyethersulfone, polyetherimide, polyethylene, polyphenylene sulfide, polyphenylene oxide, polyvinyl chloride, polytetrafluoroethylene, polypropylene, or polystyrene. In one or more embodiments, the medical utility device 10 may be manufactured by additive manufacturing.

FIG. 7 is a perspective view of a medical utility device 10 engaged with the plurality of collection tubes 12 and an extraction device 40. FIG. 8 is a perspective view of the medical device 10 engaged with the plurality of collection tubes 12 and the extraction device 40 engaged with one of the collection tubes 12. In the illustrated embodiment, the extraction device 40 includes a butterfly needle 42 and a holder 44, such as a vacuum tube holder, connected by tubing 46. The holder 44 may include a detachable connection, such as a Luer-lok™ connection, that engages with the tubing 42 and a safety valve 48 which engages with the collection tube 12. Those skilled in the art will recognize that the extraction device alternatively may include other types of needles, tubing, and/or holders.

In operation, the user inserts the plurality of collection tubes into corresponding receptacles 14 of the base 16. The user inserts the butterfly needle 42 into a patient for drawing blood. The user utilizes one hand to hold the holder 44 and uses the other hand to hold the medical utility device 10. The user engages the holder 44 and aligns the holder 44 with one of the plurality of collection tubes 12. The holder 44 is placed over the upper end of the collection tube 12 so that the safety valve 48 inserts through a rubber stopper 50 of the collection tube 12. In this way, a blood specimen is drawn through the extraction device 40 and into the collection tube 12. Once the blood specimen is collected, the user removes the holder 44 from the collection tube 12.

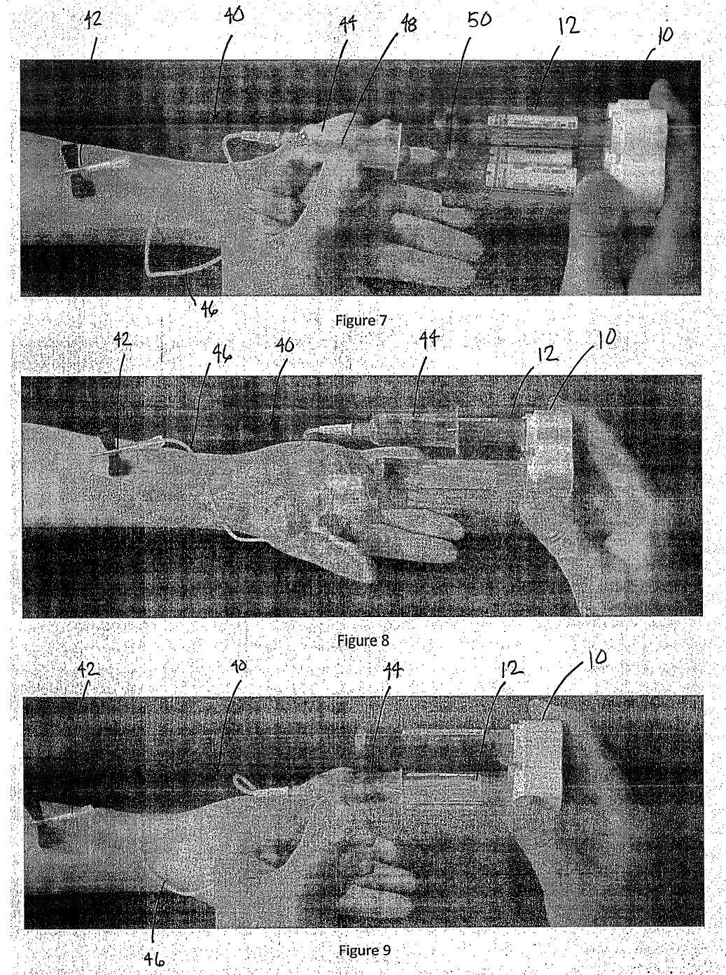

FIG. 9 is a perspective view of the medical device 10 engaged with a plurality of collection tubes 12 and the extraction device 40 engaged with another one of the collection tubes 12. The user utilizes his hand to rotate the medical utility device 10 and aligns the holder 40 with the second collection tube 12. The holder 44 is placed over the upper end of the collection tube 12 so that the safety valve 48 inserts through a rubber stopper 50 of the collection tube 12. In this way, a blood specimen is drawn through the extraction device 40 and into the collection tube 12. Once the blood specimen is collected, the user removes the holder 44 from the collection tube 12. This process is repeated until all of the collection tubes 12 contain blood specimens. However, in alternate embodiments, any number of collection tubes can contain blood specimens.

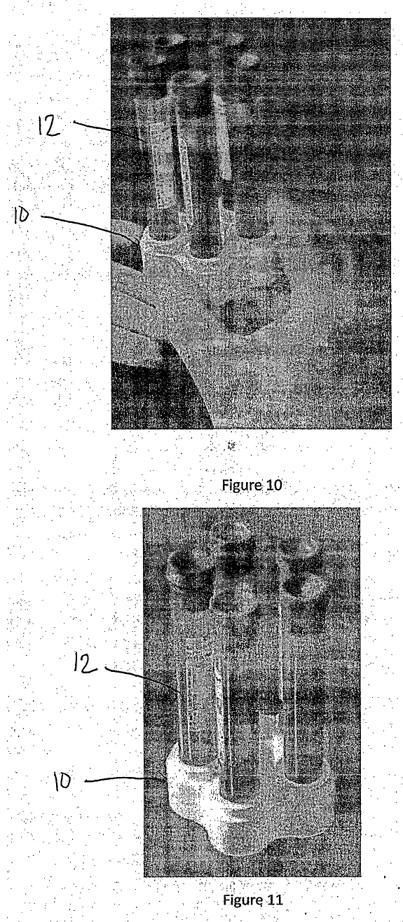

FIG. 10 is a perspective view of a user holding the medical device 10 engaged with the plurality of collection tubes 12. FIG. 11 is a perspective view of a medical device 10 engaged with the plurality of collection tubes 12 resting on a surface 52. At any time during the process of drawing the blood specimen, the user may conveniently hold the medical utility device 10 in an upright position or rest the medical utility device 10 on any flat surface 52 for storage. In this way, the medical device effectively functions as a rack for holding a plurality of collection tubes with specimens. Multiple medical utility devices 10 can be arranged in an interfacing manner for safe storing large amounts of collection tubes 12.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the subject matter set forth herein without departing from its scope. While the dimensions and types of materials described herein are intended to define the parameters of the disclosed subject matter, they are by no means limiting and are exemplary embodiments. Many other embodiments will be apparent to those of skill in the art upon reviewing the above description. The scope of the subject matter described herein should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

As used herein, an element or step recited in the singular and proceeded with the word “a” or “an” should be understood as not excluding plural of said elements or steps, unless such exclusion is explicitly stated. Furthermore, references to “one embodiment” of the presently described subject matter are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

This written description uses examples to disclose several embodiments of the subject matter set forth herein, including the best mode, and also to enable a person of ordinary skill in the art to practice the embodiments of disclosed subject matter, including making and using the devices or systems and performing the methods. The patentable scope of the subject matter described herein is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

Since certain changes may be made in the above-described systems and methods, without departing from the spirit and scope of the inventive subject matter herein involved, it is intended that all of the subject matter of the above description or shown in the accompanying drawings shall be interpreted merely as examples illustrating the inventive concept herein and shall not be construed as limiting the inventive subject matter.

Changes can be made in the above constructions without departing from the scope of the disclosure, it is intended that all matter contained in the above description or shown in the accompanying drawings shall be interpreted as illustrative and not in a limiting sense.

What is claimed is:

1. A medical utility device, comprising:
a unitary body having a base surface, an upper surface and an intermediate surface extending between the base surface and the upper surface; and
a plurality of receptacles configured to removably engage a portion of a collection tube in connection with a venipuncture procedure.

2. The medical utility device of claim 1, further comprising a hub positioned along a central axis.

3. The medical utility device of claim 1, further comprising a raised rim proximate an upper end of at least one of the receptacles.

4. The medical utility device of claim 1, further comprising a through-hole proximate a lower end of the receptacle.

5. The medical utility device of claim 1, wherein the lower surface is a generally planar surface.

6. The medical utility device of claim 1, wherein the intermediate surface comprises a perimeter having a pattern configured for interfacing with one or more additional medical utility devices.

7. The medical utility device of claim 1, wherein the intermediate surface comprises a perimeter that is generally pentagon-shaped with at least one rounded corner.

8. The medical utility device of claim 7, wherein the perimeter includes at least one indented portion disposed between the rounded corners.

9. The medical utility of claim 8, wherein a radius of the at least one rounded corner is generally equal to a radius of the at least one indented portion.

10. The medical utility device of claim 1, wherein the unitary base is produced using additive manufacturing.

11. The medical utility device of claim 1, wherein the unitary base comprises a polymer.

12. The medical utility device of claim 1, wherein the unitary body and plurality of receptacles form a rack configured for holding a plurality of collection tubes.

13. A medical utility device, comprising:
a unitary body having a base surface, an upper surface and an intermediate surface extending between the base surface and the upper surface;
a plurality of receptacles configured to removably engage a portion of a collection tube in connection with a venipuncture procedure;
wherein the plurality of receptacles are configured to engage the collection tube in an upright position;
wherein the unitary body is configured for engagement by a user in connection with a venipuncture procedure.

14. The medical utility device of claim 12, wherein the unitary body and plurality of receptacles form a rack configured for holding a plurality of collection tubes.

15. A method of storage for a plurality of collection tubes, comprising:
providing a medical utility device having a unitary body having a base surface, an upper surface and an intermediate surface extending between the base surface and the upper surface, and a plurality of receptacles configured to removably engage a portion of a corresponding collection tube in connection with a venipuncture procedure; and
collecting a blood specimen into at least one of the plurality of collection tubes.

16. The method of storage for a plurality of collection tubes of claim 15, wherein the intermediate surface comprises a perimeter having a pattern configured for interfacing with one or more additional medical utility devices.

17. The method of storage for a plurality of collection tubes of claim 15, wherein the intermediate surface comprises a perimeter that is generally pentagon-shaped with at least one rounded corner.

18. The method of storage for a plurality of collection tubes of claim 15, wherein the intermediate surface comprises a perimeter that is generally pentagon-shaped with at least one rounded corner.

19. The method of storage for a plurality of collection tubes of claim 15, wherein the intermediate surface comprises a perimeter that is generally pentagon-shaped with at least one rounded corner.

20. The method of storage for a plurality of collection tubes of claim 15, wherein the perimeter includes at least one indented portion disposed between the rounded corners.
